(19) United States
(12) Patent Application Publication
(10) Pub. No.: US 2026/0194343 A1
(43) Pub. Date: Jul. 9, 2026

(54) Method and Device for Laser Ray Tracing Measurement of Intraocular Distances and Structures

(71) Applicant: Tracey Technologies Corp., Houston, TX (US)

(72) Inventors: Vasyl Molebny, Houston, TX (US); Joe S. Wakil, Houston, TX (US); Ievgen Smyrnov, Kyiv (UA); Igor Chyzh, Kyiv (UA); Sridhar Madala, Houston, TX (US)

(73) Assignee: Tracey Technologies Corp., Houston, TX (US)

(21) Appl. No.: 19/134,294

(22) PCT Filed: Nov. 30, 2023
(86) PCT No.: PCT/US2023/081765

§ 371 Date: May 30, 2025

Related U.S. Application Data
(60) Provisional application No. 63/429,072, filed on Nov. 30, 2022.

(51) Int. Cl. G01B 11/14; G01B 9/06

(52) U.S. Cl. CPC G01B 11/14; G01B 9/06

(57) ABSTRACT

Provided herein are a method and device for laser ray tracing measurement of intraocular distances and structures. Generally, the device has a laser, pairs of acousto-optical deflectors, drivers, frequency generators, and beam splitters, a telescope and collimating lens, a basic optometric channel and a processing unit arranged and configured to probe an eye with modulated laser beams oriented to cross along optically determinable paths. Analysis of the detectable backscattered radiation is used to confirm the position of a beam cross-point and to calculate the distance thereof to an apex of the cornea. Data obtained from repeated probing is used to reconstruct the intraocular structure of the eye.

### CROSS-REFERENCE TO RELATED APPLICATIONS

This international application claims benefit of priority under 35 U.S.C. § 119(e) of provisional application U.S. Ser. No. 63/429,072, filed Nov. 30, 2022, the entirety of which is hereby incorporated by reference.

### BACKGROUND OF THE INVENTION

### Field of the Invention

The present invention relates generally to the fields of ophthalmic instruments that are used to examine the eye. More specifically, the present invention relates to ophthalmic examination instruments that measure the intraocular distances in the eye, especially related to the crystalline lens and to its position and the state of its media that is necessary for diagnosing the eye and calculating the parameters of the to-be-replaced crystalline lens.

### Description of the Related Art

With age, the quality of the crystalline lens of the human eye degrades in its opacity and refraction features inducing a dysfunctional lens syndrome. When the degree of degradation comes down to a certain level, the replacement of the natural lens may appear to be needed with an artificial intraocular lens (IOL). To appropriately define the type of the IOL, to accurately calculate its optical power and to define its position, including the distance to the apex of the cornea, the anterior chamber depth must be measured, i. e., the distance from the anterior surface of the crystalline lens to the apex of the cornea. The tilt of the lens in regards to the optical or to the visual axis of the eye is another parameter having its significance.

The depth of the anterior chamber may be evaluated using the gonioscopy with a special lens contacting the cornea, or using the ultrasonography with a special acoustic transducer. These procedures are uncomfortable for the patient and are not accurate. A solution was proposed by Schippert et al. in U.S. Pat. No. 6,631,990 to achieve better accuracy due to a three-axis linearly movable interface assembly combined with a slit lamp made operative to pick up motions of the microscope assembly in the direction of the optical axis and orthogonal directions.

The prior art also has disclosed the optical coherence tomography (OCT) for measuring the biometric variables of the eye used separately (C. Baker et al., U.S. Pat. No. 7,400,410) or in combination with topography or keratometry (R. Ebersbach et al., U.S. Pat. No. 10,694,941). The main problem arising when combining the OCT with other technologies is the “sewing” of the accurate data from the OCT imaging under the conditions of comparatively fast-moving eye.

Meanwhile, the approaches for getting the information from the space-limited volumes due to the selection of the interfering coherent light scattered in these volumes are known. The examples are represented by laser velocimetry where the scattered light from a space-limited volume of crossed laser beams is detected and is analyzed, the interfering fringes are converted into the light modulation, parameters of modulation are the measure of the processes inside that space-limited volume, the frequency of the modulation being proportional to the velocity of moving scattering particles inside the volume (R. Dandliker, et al., U.S. Pat. No. 3,895,873).

Getting the information from the space-limited volume of crossed laser beams was implemented for measurement of the cornea shape (V. Molebny et al. “High precision double-frequency interferometric measurement of the cornea shape”. Proc. SPIE 1996, Vol. 2965, pp. 121-126). According to this approach, the laser probing of the cornea is made by a doubled laser beam produced by acousto-optical modulation resulting in two frequency-shifted laser beams. The backscattered light from the cross-volume on the corneal surface is detected and analyzed to contain both beam carrier frequencies, simultaneous presence of both resulting in the difference frequency filtered after the detection, its parameters providing the information on the cross-volume.

To apply this approach for depth scanning along the Z axis in the intraocular space, the features for the laser beams, schematics, and corresponding procedures are to be found that certify the reception of the backscattered signal from a defined volume. The search for the solution of this problem is the purpose of the invention.

### SUMMARY OF THE INVENTION

The proposed solution includes the method of and the device for laser ray tracing measurement of intraocular distances and structures. The method is based on probing an eye with a combination of laser beams configured in a way to cross each other inside the eye along optically determinable paths, where the beams are produced from a single laser beam by diffraction shifting their carrier frequencies in regards to each other. The laser light scattered in the volume where the beams cross each other is detected and analyzed. The frequency shifts of laser carrier frequencies are preset in a combination enabling the confirmation of the beam crossing only when a specified frequency is filtered from the detected backscatter signals. With two beam pairs, the difference frequencies of both of them, being filtered and mixed with each other, result in the specified frequency that may be either the sum or the difference of the pair of filtered frequencies. The position and the trace of the beam cross-points inside the eye being thus defined, the distance and the media structure along the trace may be calculated, as well as the tilt of the lens.

A device implementing the proposed method consists of a laser with a pair of orthogonally deflecting acousto-optical deflectors at its output driven by drivers connected to generators, the output of the deflectors is directed into the eye through a sequence of a first telescope, a collimating lens. The generators feeding one of the deflectors generate two pairs of signals, the first pair having the first frequency difference, the second pair having the second pair of frequencies. The first and the second pairs of frequencies applied to one of the deflectors produce a pair of double-beams. Deflecting optical elements are placed in front of the eye configuring the skew paths of the double-beams directed into the eye at skew angles symmetrically to measure the distances along the optical axis. When measuring the tilts of the crystalline lens, peripheral distances are measured by directing one of the double-beams in parallel to the optical axis.

A coherent detector with a low-pass filter at its output is placed on the path of laser radiation backscattered in the eye and routed farther to the coherent detector through a beam splitter. A fluidic lens is installed on the path of the light to the coherent detector with a function to keep it conjugated with a cross-point in the process of measurement. A phase discriminator is placed at the output of the coherent detector with the low-pass filter. A reference signal for the phase discriminator is produced as a combination frequency which may be a difference or a sum of difference frequencies controlling two double-beams. A basic optometric channel also is included in the device containing a target, an optical system positioning and aligning the device, and a means for measuring the topography of the cornea and the wavefront characteristics of the eye.

### BRIEF DESCRIPTION OF THE DRAWINGS

So that the above-recited features of the invention are to be understood in detail, more particular descriptions of the invention briefly summarized above are illustrated in the appended drawings. These drawings form a part of the specification. However, that the appended drawings illustrate preferred embodiments of the invention, they are not to be considered limiting in their scope.

FIG. 1 illustrates the principle of triangulation for finding the distance by intersecting the target with two directions symmetric in points A and B.

FIG. 2 illustrates the step-by-step ray tracing of the beam-crossing points L₁, . . . Lᵢ, . . . Lₙ along the optical axis. The beams a and b are skewed symmetrically to the optical axis, being tilted at constant angles α, with distance bᵢ between the entrance points Aᵢ and Bᵢ on the cornea.

FIG. 3 illustrates the triangulation case with one of the beams AL normal to the base AB.

FIG. 4 illustrates the step-by-step ray tracing of the beam-crossing points Lₐ₁, . . . Lₐᵢ, . . . Lₐₙ along the beam a hitting the eye in parallel to the optical axis.

FIG. 5 is a functional schematic of the device embodiment with the means for probing the eye with laser beams, for detecting the backscattered light, converting it into signals which are processed and from which the information on intraocular distances and structures is derived.

FIG. 6 shows the crossings of a potential set of laser beams enabling the measurements of ocular distances in both, X0Z and Y0Z planes.

FIG. 7 demonstrates the compatibility of measurement of intraocular distances in X0Z and Y0Z planes and of the wave front using the central part of the beam paths.

FIG. 8 illustrates the paths of the beams in the schematic of FIG. 5 bent by pairs of mirrors M₃, M₄ and M₅, M₆ for a case of axial distance measurement using the symmetrically tilted laser beams as configured in FIGS. 1 and 2.

FIG. 9 illustrates the spectra of the laser beams projected into the eye. Double-beams a have the carrier frequencies fₐ₁ and fₐ₂ with their difference Fₐ. Double-beams b have the carrier frequencies f_{b1} and f_{b2} with their difference F_{b}. For the sake of symmetry of beam tilts, the frequencies fₐ₁ and f_{b1} (or, fₐ₂ and f_{b2}) are chosen to be equal to each other. Shift frequencies (or, difference frequencies) Fₐ and F_{b} are much smaller than the carrier frequencies fₐ and f_{b}. The frequency f₀ is the central frequency of the first order of diffraction in both X and Y deflectors. It is important that Fₐ is not equal to F_{b}. Filled circlets and the solid lines designate the active beams (probing the eye sequentially in time). Empty circlets and the dashed lines designate the switched-off (inactive) beams.

FIG. 10 illustrates the paths of the double-beam a parallel to the optical axis and the double-beams b bent by the pair of mirrors M₅, M₆ for a case of distance measurement by triangulation as configured in FIGS. 3 and 4. A mirroring configuration takes place if the double-beams a are tilted by mirrors or M₃, M₄ and the double-beam b is parallel to the optical axis.

FIG. 11 illustrates the spectra of the laser beams projected into the eye for the case shown in FIG. 10. The double-beam a has carrier frequencies fₐ₁ and fₐ₂, constant during the cycle of measurement. The frequencies f_{b1} and f_{b2} forming the double-beams b are changing in the course of measurement with their difference Fb being constant. Frequency Fₐ is not equal to frequency F_{b}.

FIG. 12 illustrates the process of accumulation of the signals showing the structure of the eye (on the example of the crystalline lens) during a session of measurements starting at the moment t₁ when the double beam a and the double beam b cross each other at her distance h₁ from the apex of the cornea (corresponding to the anterior surface of the lens) and finishing with the distance hₙ from the apex of the cornea (corresponding to the posterior surface of the lens). The values of the beam frequencies for the time moment t₁ are designated as {fₐ₁ₐ₂ & f_{b1b2}}ₜ₁. The designation {fₐ₁ₐ₂} means that the double beam a has two carrier frequencies fₐ₁ and fₐ₂. Similarly, the double beam b with the carrier frequencies designation {f_{b1b2}} has two carrier frequencies f_{b1} and f_{b2}. Cumulatively, in the time moment t₁, the frequencies of the beams a and b are designated as {fₐ₁ₐ₂ & f_{b1b2}}ₜ₁. The subscripts at the brackets of this designation are changed to tᵢ and tₙ for the time moments tᵢ and tₙ respectively.

### DETAILED DESCRIPTION OF THE INVENTION

As used herein, the term “a” or “an” when used in conjunction with the term “comprising” in the claims and/or the specification may mean “one”, but it is also consistent with the meaning of “one or more”, “qt least one”, and “one or more than one”. Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or”.

As used herein, “comprise” and its variations, such as “comprises” and “comprising”, will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the terms “consists of” and “consisting of” are used in the exclusive, closed sense, meaning that additional elements may not be included.

As used herein, the ordinal adjectives “first”, “second”, “third”, and “fourth” unless otherwise specified are used to describe a common object, merely indicate that different instances of like objects are being referred to, and are not intended to imply that the objects so described must be in a given sequence, either temporally, spatially, in ranking, or in any other manner.

As used herein, the term “includes” or “including” is used herein to mean “including, but not limited to”. The terms “includes”, “including” and “including, but not limited to” are used interchangeably.

In one embodiment of the present invention there is provided a method for laser ray tracing measurement of intraocular distances and structures, comprising probing an eye with a laser beam by directing simultaneously at least two modulated laser beams into the eye, said modulated laser beams oriented to cross each other along optically determinable paths; detecting the backscattered laser radiation and analyzing it to contain a combination of both modulated frequencies, thereby confirming the position of a beam cross-point; calculating the distance of the beam cross-point to an apex of the cornea; repeating the step of probing the eye by varying traces of the cross-points in the eye, storing data obtained thereby and reconstructing an intraocular structure of the eye, and performing the step of probing the eye in two separate sessions in orthogonal planes containing the optical axis.

In this embodiment the method may comprise single-sideband modulating of each of said laser beams by shifting a carrier frequency of a first laser beam by a first difference frequency transforming it into a first laser double-beam, and by shifting the carrier frequency of a second laser beam by a second difference frequency transforming it into a second laser double-beam; confirming the detection of the beam cross-point by detecting and filtering a combination of both difference frequencies from the back scattered laser radiation from the eye, said combination comprising a sum or a difference of said difference frequencies calculating the distance of the beam cross-point to the apex of the cornea; repeating the step of probing the eye by varying the traces of the cross-points in the eye, storing the data from the repeated procedures and reconstructing the intraocular structure of the eye, and performing the step of probing the eye in two separate sessions in orthogonal planes containing the optical axis. Further to this embodiment the method may comprise deriving optically determinable paths from a corneal topography and the ray tracing wavefront measurement. In one aspect of these embodiments the first laser double-beam and the second laser double-beam may be symmetrically tilted with reference to the visual axis of the eye. In another aspect the first laser double-beam may be directed coincident with or in parallel to the visual axis of the eye; where the second laser beam is tilted.

In another embodiment of the present invention there is provided a device for laser ray tracing measurement of intraocular distances and structures in an eye, comprising a laser configured to emit a laser beam of a wavelength suitable for ray tracing; a pair of acousto-optical deflectors comprising a first deflector configured to deflect said laser beam in a first plane containing an optical axis and a second deflector configured to deflect said laser beam in a second plane containing the optical axis and orthogonal to the first plane, each acousto-optical deflector in the pair having an entrance aperture and an exit aperture and each positioned along a path of the laser beam whereby effective centers of deflection thereof substantially coincide; a first driver with an output operably connected to the first deflector, and a second driver with an output operably connected to the second deflector; a first frequency generator with an output connected to the first driver, and a second frequency generator with an output connected to the second driver; a telescope and a collimating lens placed sequentially in optical alignment with the centers of deflection of said pair of acousto-optical deflectors; a first beam splitter placed in the path of the laser beam to the human eye and of the back scattered laser radiation from the eye; a second beam splitter placed after the first beam splitter on the path to the eye; a basic optometric channel placed with an optical axis bent to the eye by the second beam splitter, said basic optometric channel containing a target, a system of positioning and alignment of the device, and means for measuring a topography of the cornea and wavefront characteristics of the eye; and a processing unit with a display, said processing unit in operable communication with said laser, with said frequency generators, and with said basic optometric channel. Where in this embodiment the laser beam propagation zone is divided into an internal zone and an external zone, of which the internal zone is configured for ray tracing the eye with the laser beams parallel to the optical axis while the external zone is configured for oblique ray tracing of the eye; a set of optical elements is placed in the external zone directing the probing laser beams at skew angles; the structure of each of said first frequency generator and said second frequency generator comprises a first oscillator of a first frequency, a second oscillator of a second frequency, a third oscillator of a third frequency, a fourth oscillator of a fourth frequency, and a combination-frequency unit, said first frequency and said second frequency having a first frequency shift between them that defines a spatial splitting in a first double-beam, said third frequency and said fourth frequency having a second frequency shift between them that defines a spatial splitting in a second double-beam, said combination-frequency unit combining said first frequency shift and said second frequency shift as a difference therebetween or as a sum thereof, and a configuration of the first frequency generator and the second frequency generator enabling a mode of all oscillators functioning simultaneously or a mode of a single functioning oscillator. Where in this embodiment a coherent detector is placed on the path of the back scattered laser radiation after the first beam splitter with an aperture in front of said coherent detector; a low-pass filter is placed at the output of the coherent detector, said low-pass filter being connected to the processing unit; and a phase discriminator is placed after the low-pass filter with its signal input connected to the output of said low-pass filter, and its reference input having when-enabled connections to the combination-frequency units of said first frequency generator or said second frequency generator, the output of said phase discriminator connected to said processing unit; said processing unit in its connections with said first frequency generator and said second frequency generator are configured to provide measurement in said first plane and said second plane separately to enable the connection of said first frequency, said second frequency, said third frequency and said fourth frequency from said first frequency generator to said first driver, the connection of a single frequency from said second frequency generator to said second driver, the connection of the combination frequency from said first frequency generator to the reference input of said phase discriminator when providing the measurement in the first plane, and to enable the connection of said first frequency, said second frequency, said third frequency and said fourth frequency from said second frequency generator to said second driver, the connection of a single frequency from said first frequency generator to said first driver, the connection of the combination frequency from said second frequency generator to the reference input of said phase discriminator when providing the measurement in the second plane.

In one aspect of this embodiment each of the first frequency generator and the second frequency generator may be configured as a first four-channel frequency synthesizer and a second four-channel frequency synthesizer with synchronous direct digital synthesis. In another aspect the first frequency generator and the second frequency generator may be configured as a single eight-channel frequency synthesizer with synchronous direct digital synthesis, where its first four channels function as the first frequency generator, and its second four channels function as the second frequency generator. In this embodiment and aspects thereof the set of optical elements placed in the external zone comprises a first set of mirrors and a second set of mirrors, each of said set of mirrors consisting of two outward reflecting mirrors, symmetrically placed in reference to the optical axis, and two symmetrically placed inward reflecting mirrors, the first set of mirrors configured to bend the optical paths in the first plane, the second set of mirrors configured to bend the optical paths in the second plane.

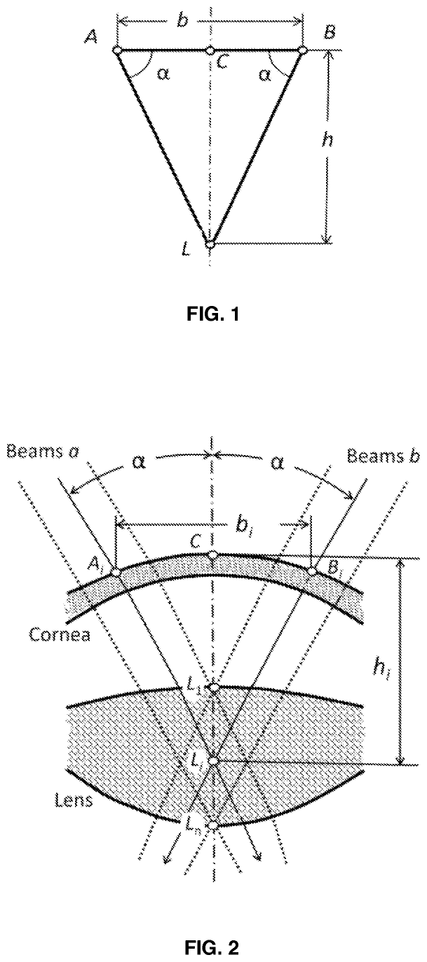

The principle of triangulation is applied in this invention to measure the intraocular distances. The geometric problem is easily resolved for two beams AL and BL passing through two points A and B with known coordinates and crossing each other in a third point L. FIG. 1 illustrates a case when the angles towards point L in points A and B are the same and equal to α. Distance h for this case can be defined as h=(b/2)×tan α. When the paths of the beams are routed through different media, the task is still resolvable with certain a priori knowledge and suggestions related to these media. In the case of the human eye, these media are the cornea, the crystalline lens, the anterior and posterior chambers. The preliminary information on said media can consist of known eye models, measured corneal topography and wavefront. All this information relates to a prior art, and will not be the object of discussion in the proposed invention.

FIG. 2 illustrates the step-by-step ray tracing of the beam crossing-points along the optical axis of the eye. For the sake of simplification, the visual and the optical axes of the eye are suggested to coincide. Also, laser beams are presented in the geometrical drawings as lines, no attention is paid to the size of their cross-sections, including the beam doubling, wave length, or divergence.

As mentioned above, the beams a and b are skewed symmetrically to the optical axis, tilted at constant angles α, with the equal distances bᵢ from the axis to the entrance points Aᵢ and Bᵢ in the cornea. The beams are crossing each other inside the eye along the optical axis in points Lᵢ (for the sake of simplification, shown are only three points across the crystalline lens: L₁, . . . Lᵢ, . . . Lₙ) at distances hᵢ to the apex C of the cornea. The height of the point C above the points A and B is included into calculations from the known curvature of the cornea.

The points L₁, . . . Lᵢ, . . . Lₙ of beam crossings are identified using the features provided herein, their positions along the paths inside the eye are calculated and used for farther calculations to define the parameters of the intraocular lens to replace the crystalline lens and to position it correctly in the process of surgery. These calculations are not the topic of this invention.

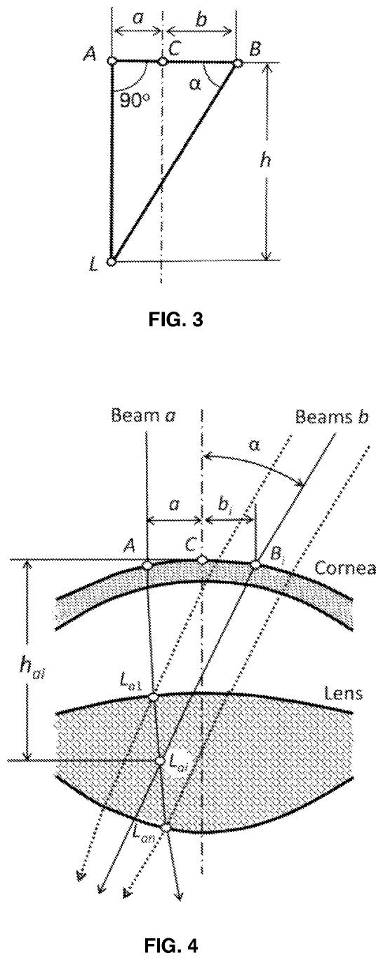

With the invention provided herein, the intraocular distances may be measured not only along the optical axis, but also along any line designated by a laser beam. In this way, the tilt of the crystalline lens may be determined by measuring the distances from the corneal surface at the periphery of the lens. FIG. 3 shows the case where one of the angles at the base AB is 90 degrees. The distance h is measured along the beam coinciding with the line AL. The distance h is calculated as h=(a+b)×tan α.

FIG. 4 demonstrates the time sequence of operations with a peripheral beam a, parallel to the optical axis unmoved during the cycle of measurements, while the beam b changes its position from interval to interval. The beams cross each other inside the eye along the path of the beam a in points Lₐᵢ (Lₐ₁, . . . Lₐᵢ, . . . Lₐₙ) at distances h, to the apex C of the cornea. Similarly, as in the symmetric case of beam tilts, the height of the point C above the points A and B is included in calculations from the known curvature of the cornea.

Measurements in each of the planes XOZ and YOZ is made separately keeping the central frequency f₀ of the orthogonal plane out of the range of interference with the other frequencies.

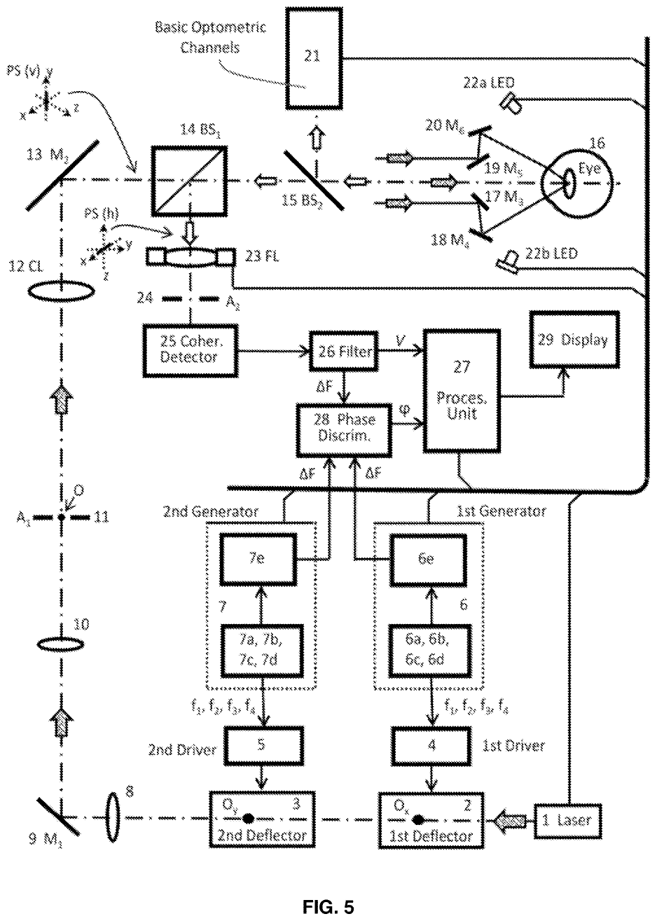

Specific features of the invention are explained herein by reference to the functional schematic of the device (FIG. 5). The laser 1 is attached by its output to the input of the first deflector 2, having its center of scanning in point Oₓ. The second deflector 3 is mounted in series with the first deflector 2. The second deflector 3 has its center of scanning O_{y}. The first deflector 2 and the second deflector 3 may be of any type, but from the consideration of the speed of deflection and scanning, acousto-optical deflectors may be recommended based on the principles of light diffraction on the grating, created in acousto-optical crystals by the ultrasound waves. For the purposes of this invention, the centers of scanning in the orthogonal (X and Y) directions coincide. Practically, the deflectors 2 and 3 may be designed in such a way to provide their crystals as near to each other as possible, on the order of millimeters. To create diffraction gratings in the crystals, drivers are used where the first driver 4 connected to the first deflector 2 is for deflecting the laser beam in the Y direction, and the second driver 5 connected to the second deflector 3 is for deflecting the laser beam in the X direction.

The angle of deflection depends on the grating spacing, defined by the frequency of the high-frequency signal applied to the transducer exciting the elastic wave in the crystal. This frequency is usually in, but not limited by, the range 60-80 MHz for deflecting the laser beam in the near infrared. Only the first order of diffraction for both, X and Y directions, is used in the device provided herein.

The selection of the first order of diffraction is made by a spatial filter, physically it is an aperture in a non-transparent material. The selected first-order beam is scanned within the selecting aperture to create a sequence of directions for probing the eye.

The drivers 4 and 5 are driven by frequency generators that generate sinusoidal voltages of certain frequencies. The first driver 4 is connected to the output of the first generator 6, while the second driver 5 is connected to the output of the second generator 7.

The first generator 6 feeds the first driver 4 with four different frequencies f₁, f₂, f₃, f₄ simultaneously when measuring the distances and structures in XOZ plane. In the course of these measurements, these four frequencies are changed as will be described below. The second generator 7 feeds the second driver 5 with only one frequency f₀ to keep the beam in the plane XOZ. The frequency f₀ is the central frequency of the first order of diffraction in both X and Y deflectors.

Similarly, the second generator 7 feeds the second driver 5 with four different frequencies f₁, f₂, f₃, f₄ simultaneously when measuring the distances and structures in YOZ plane. In this cycle of measurements, the four frequencies are also changed from probing to probing, but the first generator 6 feeds the first driver 4 with only one frequency f₀ during the whole cycle to keep the beam in the plane YOZ. The value of f₀ is ascribed to any of four frequencies f₁, f₂, f₃, f₄.

At the periphery of the paths of laser beams in XOZ and YOZ planes, optical components are placed, bending the laser beams twice: outwards, and then—inwards (FIG. 5). These components may be small-width mirrors, since measurements of intraocular distances and structures are performed only in the planes XOZ and YOZ with laser beam diameter not exceeding 1 mm. Beam tilting, necessary for triangulation, is performed by two sets of mirrors 17-18 (M₃-M₄) and 19-20 (M₅-M₆) for each plane.

For easier alignment, the pairs of mirrors 17-18 (M₃-M₄) and 19-20 (M₅-M₆) may be implemented as faces of a pyramid or, as conical mirror surfaces of an axicon.

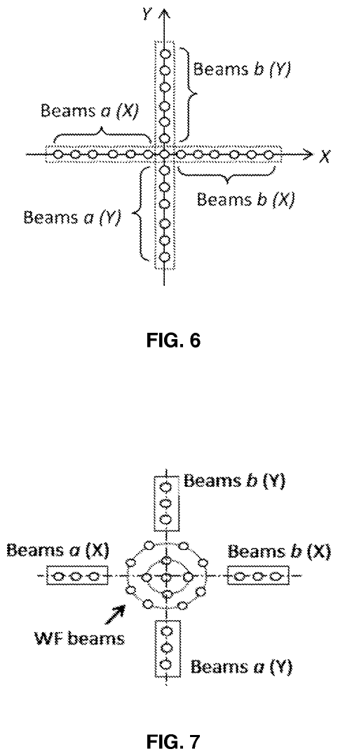

A layout of beam cross-sections at the entrance in the eye is demonstrated in FIG. 6. For convenience of further description, the left and the lower wings of the layout are designated as “a”-beams, the right and the upper wings are designated as “b”-beams. Their number (density of beams) along the X and Y axes depends on the required spatial resolution of structure measurement. The laser ray tracing procedures of this invention are compatible with the procedures of wave front measurements requiring the use of the central zone of beam paths that is easily configurable as shown in FIG. 7.

The task of four-frequency generators is to form two pairs of laser beams (or, in other words, a pair of double-beams). For example, a first pair f₁, f₂ forms an “a” double-beam, and a second pair f₃, f₄ forms a “b” double-beam. Connotating the frequencies by the beams which they form, the frequencies f₁, f₂, f₃, f₄ are designated as fₐ₁=f₁, fₐ₂=f₂, f_{b1}=f₃, f_{b2}=f₄. Difference frequencies of these pairs are F₁=Fₐ=f₁−f₂ and F₂=F_{b}=f₃−f₄. The frequencies F₁ and F₂ (farther called also shift frequencies) are much smaller than the frequencies f₁, f₂, f₃, f₄. It is important that F₁ is not equal to F₂. The designation of frequencies fₐ₁ and fₐ₂ with their difference Fₐ and frequencies f_{b1} and f_{b2} with their difference F_{b} are used for both, XOZ and YOZ planes.

Embodiments of the first frequency generator 6 and of the second frequency generator 7 are the same. Each of them contains four oscillators generating certain frequencies feeding the drivers 4 and 5 respectively: a first oscillator 6a generates a first frequency f₁, a second oscillator 6b generates a second frequency f₂, a third oscillator 6c generates a third frequency f₃, a fourth oscillator 6d generates a fourth frequency f₄. The same frequencies f₁, f₂, f₃, f₄ are generated by a first (7a), a second (7b), a third (7c), and a fourth (7d) oscillators of the second generator 7. The frequency generator 6 contains also a combination-frequency unit 6e connected to the oscillators 6a, 6b, 6c, and 6d. Similarly, the frequency generator 7 contains also a combination-frequency unit 7e connected to the oscillators 7a, 7b, 7c, and 7d. Each of these combination-frequency units (6e and 7e) delivers a combination of frequencies from mentioned oscillators, that are either [(f₁−f₂)−(f₃−f₄)], or [(f₁−f₂)+(f₃−f₄)]. For distinctiveness, a designation of the frequency difference ΔF=[(f₁−f₂)−(f₃−f₄)] is used in the drawings.

The drivers 4 and 5 can function in two modes: in the mode of wave front measurements and in the mode of measurement of intraocular distances and structures. In the mode of wave front measurements, the first driver 4 and the second driver 5 control the deflectors 2 and 3 by only one frequency each, for example, by the first frequency f₁, which, in this mode, will be fₓ₁ for X channel, and f_{y1} for Y channel.

The mode of measurement of intraocular distances and structures consists of two procedures, a first procedure is performed for the XOZ plane, and a second procedure for the YOZ plane. During the first procedure, the second deflector 3 keeps the beam non-split in Y direction, while the first deflector 2 splits the input laser beam in two pairs of double beams, the frequencies f₁, f₂, f₃, f₄ having the interrelations as described above. During the second procedure, the first deflector 2 keeps the beam non-split in X direction, while the second deflector 3 splits the input laser beam in two pairs of double beams, the frequencies f₁, f₂, f₃, f₄ having the same interrelations, also described above.

The laser light output from the second deflector 3 is directed into the telescope created by two lenses 8 (L₁) and 10 (L₂). The path of laser beams into the eye is connoted by the cross-hatched arrows. To make the design more compact, the optical path between the lenses 8 and 10 is bent by the mirror 9 (M₁ ). The centers of scanning Oₓ and O_{y} of each of the deflectors 2 and 3 are transferred into the space after the lens 10. In the assumption that Oₓ and O_{y} are managed to coincide or to be at so small distance between them, that it may be neglected, the center of scanning after the lens 10 is designated as O, coinciding with the back focus of the lens 10. In the space of diffracted beams, its position must coincide with the direction of the first order of diffraction, both in X and in Y directions. Spatial filtering, selecting the first order of diffraction, is provided by the aperture 11 (A₁), a hole, which size is smaller than the distance between the orders of diffraction, practically, its diameter is of the order of a millimeter. The center of this aperture coincides with the point O.

The next optical element on the path of the laser beam is the collimating lens 12 (CL). Its front focus coincides with the back focus of the lens 10, and as such, with the center of scanning O in the center of the aperture 11 (A₁), playing the role of a spatial filter-selector of the first order of diffraction, and creating parallel beams at the exit of the lens 12.

Mirror 13 (M₂) bends the beam direction towards the first beam splitter 14 (BS₁), and after passing through the second beam splitter 15 (BS₂), towards the eye 16. The first bean splitter 14 (BS₁) preferably is a polarizing beam splitter. It enables the vertical linear polarization to continue its path to the eye. The role of the first beam splitter 14 also consists in reflecting the component of the polarization of the light backscattered from the eye, orthogonal to the initial laser polarization. These polarization features are illustrated by the insets near the beam splitter 14 showing the polarization states of the laser “light-in” and the scattered laser “light-out”.

The reciprocal positioning of the device and of the patient's eye and any other necessary alignments, are performed using a basic optometric channel 21. For these purposes, Purkinje reflexes are provided by several light emitting diodes (LEDs), two of them shown in FIG. 5, are 22a and 22b. The beam splitter 15 reflects the light scattered back from the eye, including these Purkinje images. Being known in the art, the optometric details, not related to the design or principles of the invention herein, are not discussed herein.

For detection of laser beams interfering inside the eye, a coherent detector 25 is installed on the path of the light (connoted by empty arrows) from the eye 16 going through beam splitter 15, bent by beam splitter 14, through fluidic lens (FL) 23, and aperture 24 (A₂ ). The coherent detector 25 is connected at its output to a low-pass filter 26, that is adjusted for passing the frequency ΔF which is equal to the difference of two frequencies: ΔF=Fₐ−F_{b}. The coherent detector, as a non-linear component, combines the frequencies f₁, f₂, f₃, f₄. Taking into account that the frequencies f₁, f₂, f₃, f₄ are of the order of 60 MHz, the difference between any of f₁, f₂ and any of f₃, f₄ is of the order of MHz, the selection of the differences F₁=f₁−f₂ and F₂=f₃−f₄ is an easy task, since it may be much lower. This means that only frequencies F₁=f₁−f₂ and F₂=f₃−f₄ are used in producing their difference ΔF=F₁−F₂. As mentioned in the description of frequency generators 6 and 7, a combination of a sum of F₁ and F₂ also is possible.

The low-pass filter 26 has two outputs. One of them is connected to the processing unit 27, by the amplitude V of the signal of the frequency ΔF delivering the information to the processing unit on the level of scatter in the zone of beam crossings a and b. The other output is connected to the signal input of a phase discriminator 28. The reference input of said phase discriminator 28 is connected to the combination-frequency units 6e and 7e, each of them able to deliver the reference signal of the frequency ΔF when getting the permission from the processing unit 27. When measurements of intraocular distances and structure are made in the first plane XOZ, then signal from the combination-frequency unit 6e is enabled. When measurements of intraocular distances and structure are made in the second plane YOZ, then the permission is given to the signal from the combination-frequency unit 7e.

The output of the phase discriminator 28 contains information on the phase o between the initial oscillations of the frequency ΔF from the output of the filter 30 and the signal of the same frequency derived from the combination of all four frequencies interfering in the cross point in the eye that is delivered to the phase discriminator 28 from the filter 26. It is noted that the frequency ΔF may be extracted from the combination of detected signal only, if both frequencies F₁ and F₂ are present in the signal, i. e., only when the signal comes from the zone of intersection of beams a and b. This phase difference reveals the information on the refraction non-homogeneity met by laser beams on the path to the cross point. In the process of measurement, the paths change, the non-homogeneity varies, and when compared trace-by-trace in the process of measurement, it can show how much the structure of refraction non-homogeneity can influence the quality of the image formed on the retina. Statistical analysis of this non-homogeneity may be done not taking into account the spreading of beam paths caused by the cornea. An important matter to be taken into account is still the traces to be densely neighboring each other, not to get more than 360-degree jump from trace to trace.

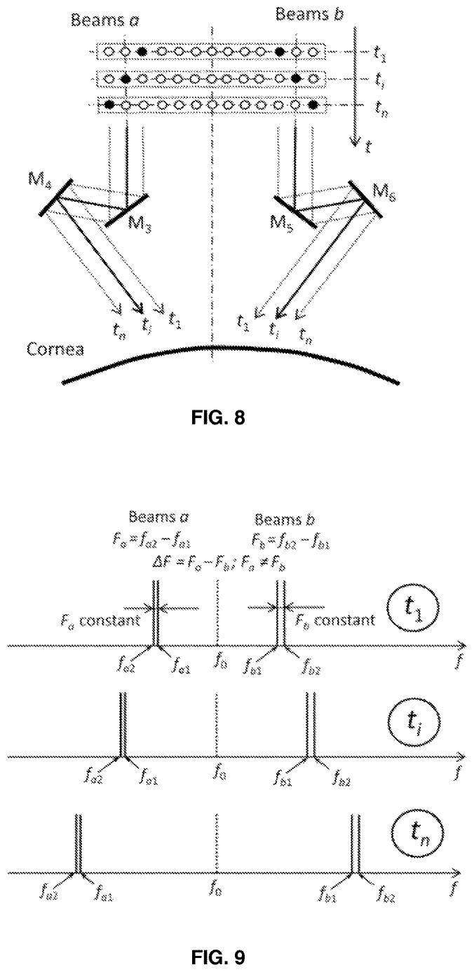

The beam paths in the device are illustrated by FIG. 8 for the case of triangulation shown in FIG. 1 when the distance measurement is performed along the optical axis as shown in FIG. 2. Because the procedures of measurement in the planes XOZ and YOZ are identical, only one of them, for the plane XOZ is described herein. After the axis of symmetry has been found and the refraction parameters have been determined, deflector 3 is set to keep the value of the coordinate Y equal to zero by applying the frequency f₀ during the whole cycle of measurement of distances along the optical axis. Meanwhile, deflector 2 is fed by four frequencies simultaneously, a pair fₐ₁ and fₐ₂ forming the double beam a, and a pair f_{b1} and f_{b2} forming the double beam b. Both pairs hit the aperture of the eye simultaneously, entering the eye tilted as shown in FIG. 2, the tilt being the result of reflections of the beams aᵢ by the mirrors M₃, M₄, and of the beams bᵢ—by the mirrors M₅, M₆. During a short interval of time tᵢ, typically, less than 10 μs, deflector 3 keeps both double beams (a₁and bᵢ) in an i-th position. During this time interval, coherent detector 25 determines the level of the signal having the frequency ΔF. In the next time interval, the frequencies fₐ₁−fₐ₂ and f_{b1}−f_{b2} are changed to set the beams a and b in the (i+1)-th position. In FIG. 8, consecutive positions during the time intervals are shown from 1 to n. The initial time interval t₁ and the end time interval tₙ for getting the information from the cross points along the optical axis, may be set in front and behind the intraocular lens. The frequency differences Fₐ and F_{b} are kept constant during the whole cycle (from the 1-st time interval to the last, n-th interval). The angle α is also kept constant during the whole cycle due to the parallel paths of the beams after the collimating lens 12. Sectioning of the beams is shown in FIG. 8 as circlets, filled-in when the beams are switched-on during an i-th interval and empty for the beams that are switched-off during this interval. The level of the signal at the exit of the coherent detector 25 as a function of time-interval sequence shows the profile of the light scatter in the ocular media along the optical axis. The phase difference at the exit of phase discriminator 28, under the condition of phase non-discontinuity in the time interval sequence, may serve as a vernier in measurement of intraocular distances.

Dynamics of the spectra in the course of measurements with the configuration of FIG. 8 is illustrated by FIG. 9. During any of the time intervals t₁, . . . tᵢ, . . . tₙ, both double beams keep constant the frequency differences Fₐ=fₐ₁−fₐ₂ and F_{b}=f_{b1}−f_{b2}, as well as keep constant the frequency difference ΔF=Fₐ−F_{b}, and the condition is kept Fₐ≠F_{b}.

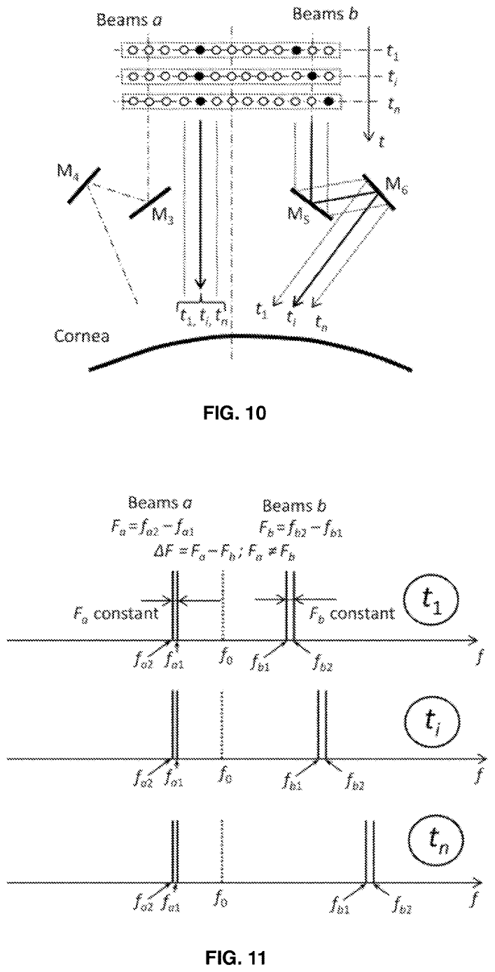

With the instant invention, the intraocular distances may be measured not only along the optical axis, but also along any line designated by a laser beam. In this way, the tilt of the crystalline lens is determined by measuring the distances, from the corneal surface, at the periphery of the lens. FIG. 10 shows the case demonstrated by FIG. 3 with one of the angles at the base AB equal to 90 degrees. The distance h is measured along the beam coinciding with the line AL. This line coincides with the optical axis, or, as shown in FIG. 4, may be at some distance from it.

FIG. 10 demonstrates the time sequence of the beam a, parallel to the optical axis unmoved during the cycle of measurements, while the beam b changes its position from interval to interval during the same cycle of measurements. Beam crossings are shown in FIG. 10 with the same designations as in FIG. 8. The circlets are filled-in when the beams are switched-on during an i-th interval and empty for the beams that are switched-off during this interval.

Spectral composition is explained in FIG. 11. It differs from the spectral composition of FIG. 9 by constant values of frequencies fₐ₁ and fₐ₂. Only beams b are scanned to cross the beam a at different depths (distances from the cornea).

To measure the tilt of the crystalline lens, the distances are measured in two orthogonal planes XOZ and YOZ. The distances from the cornea to the points Lₐ₁ (FIG. 4) measured symmetrically in the opposite peripheral zones of beam paths give a solution when made in both planes XOZ and YOZ. Other combinations of measured distances in both planes are used for calculation of the tilt. The intraocular distances are measured or calculated in regards to the apex of the cornea. Determining of the apex may be provided by corneal topography, usually combined with the wave front measurements. When topography and wave front are measured, the correction coefficient is calculated or simply found from the diagrams reconstructed with the ray tracing optical programs like Zemax. This correction coefficient defines how shorter physically is the distance inside the eye in comparison to the distance in the air. These diagrams are preprogrammed and available as libraries in the processing unit 27.

The functions of the oscillators 6a, 6b, 6c, 6d, and of the combination-frequency unit 6e, as well as the functions of the oscillators 7a, 7b, 7c, 7d, and of the combination-frequency unit 7e, may be implemented using the principles and technology of direct digital synthesis (DDS). Two pairs of software-controlled frequencies, each pair with constant difference, may be produced in a chip, such as AD 9958 or AD 9959, whose DDS cores provide independent frequency, phase, and amplitude control on each of two or four channels with the provision to phase synchronize multiple chips. Thus, control of both axes, X and Y, may be performed in two or more chips.

When measuring the intraocular distances, the sensitive surface of the coherent detector 25 must be conjugated with points (microvolumes) of beam crossings. This means that the control voltage of fluidic lens 23 is synchronized with the laser beam positions during the cycle of distance measurement. The synchronous conjugation is performed via a bus communication of the lens 23 with the processing unit 27. Necessary control commands and requested feedback also are administered from the processing unit 27 to laser 1, frequency generators 6 and 7, basic optometric channels 21, light emitting diodes 22. Information, received from the components of the device after its processing is displayed on the display 29.

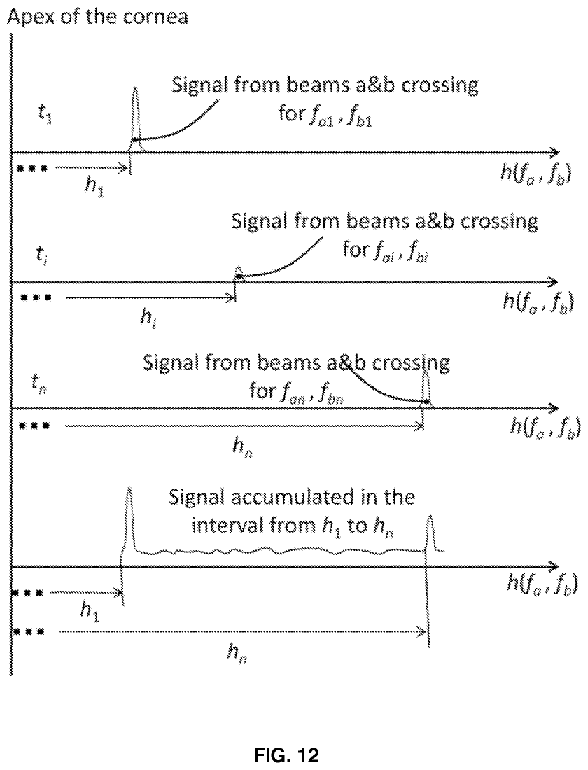

The procedure of signal accumulation is illustrated by FIG. 12. Using the triangulation approach, and based on the configuration of the optical system, the set of beam frequencies fa1, fa2 (designated as fa1a2) and fb1, fb2 (designated as fb1b2) for double bean a and for double bean b is established. These frequencies define the optical paths of both double beams and the location of their cross point. Detection of the signal of the frequency difference ΔF=Fa−Fb (where Fa=fa1−fa2 and Fb=fb1−fb2) confirms the detection from the cross point. In FIG. 12 illustrated are three points along the optical axis: the first cross point is for the time moment t1, when the set of frequencies {fa1a2, fb1b2}t1 is generated, and the coherent detector 25 (see FIG. 5), after filtering the frequency ΔF in the filter 26, outputs the signal V to the processing unit 27. The same procedure is applied in the time moments ti and tn with the set of frequencies {fa1a2, fb1b2}ti and {fa1a2, fb1b2}tn respectively. The amplitude of the signals depends on the level of signal scattering in the cross point thus defining the edges of the media and surfaces being crossed. In the case of FIG. 12, they are the anterior surface of the crystalline lens (for t1), internal medium of the lens (for ti), and posterior surface of the lens (for tn). All these data are accumulated in the processing unit 27 and can be displayed in any convenient presentation, for example, as signal variations along the trace of cross points.

What is claimed is:

1. A method for laser ray tracing measurement of intraocular distances and structures, comprising:
probing an eye with a laser beam by directing simultaneously at least two modulated laser beams into the eye, said modulated laser beams oriented to cross each other along optically determinable paths;
detecting backscattered laser radiation and analyzing it to contain a combination of both modulated frequencies, thereby confirming the position of a beam cross-point;
calculating the distance of the beam cross-point to an apex of the cornea;
repeating the step of probing the eye by varying traces of the cross-points in the eye, storing data obtained thereby and reconstructing an intraocular structure of the eye, and
performing the step of probing the eye in two separate sessions in orthogonal planes containing the optical axis.

2. The method of claim 1, comprising:
single-sideband modulating of each of said laser beams by shifting a carrier frequency of a first laser beam by a first difference frequency transforming it into a first laser double-beam, and by shifting the carrier frequency of a second laser beam by a second difference frequency transforming it into a second laser double-beam;
confirming the detection of the beam cross-point by detecting and filtering a combination of both difference frequencies from the back scattered laser radiation from the eye, said combination comprising a sum or a difference of said difference frequencies;
calculating the distance of the beam cross-point to the apex of the cornea;
repeating the step of probing the eye by varying the traces of the cross-points in the eye, storing the data from the repeated procedures and reconstructing the intraocular structure of the eye, and
performing step of probing the eye in two separate sessions in orthogonal planes containing the optical axis.

3. The method of claim 2, further comprising deriving optically determinable paths from a corneal topography and the ray tracing wavefront measurement.

4. The method of claim 2, wherein the first laser double-beam and the second laser double-beam are symmetrically tilted with reference to the visual axis of the eye.

5. The method of claim 2, wherein the first laser double-beam is directed coincident with or in parallel to the visual axis of the eye; wherein the second laser beam is tilted.

6. A device for laser ray tracing measurement of intraocular distances and structures in an eye, comprising:
a laser configured to emit a laser beam of a wavelength suitable for ray tracing;
a pair of acousto-optical deflectors comprising a first deflector configured to deflect said laser beam in a first plane containing an optical axis and a second deflector configured to deflect said laser beam in a second plane containing the optical axis and orthogonal to the first plane, each acousto-optical deflector in the pair having an entrance aperture and an exit aperture and each-positioned along a path of the laser beam whereby effective centers of deflection thereof substantially coincide;
a first driver with an output operably connected to the first deflector, and a second driver with an output operably connected to the second deflector;
a first frequency generator with an output connected to the first driver, and a second frequency generator with an output connected to the second driver;
a telescope and a collimating lens placed sequentially in optical alignment with the centers of deflection of said pair of acousto-optical deflectors;
a first beam splitter placed in the path of the laser beam to the human eye and of the back scattered laser radiation from the eye;
a second beam splitter placed after the first beam splitter on the path to the eye;
a basic optometric channel placed with an optical axis bent to the eye by the second beam splitter, said basic optometric channel containing a target, a system of positioning and alignment of the device, and means for measuring a topography of the cornea and wavefront characteristics of the eye; and
a processing unit with a display, said processing unit in operable communication with said laser, with said frequency generators, and with said basic optometric channel;
wherein:
the laser beam propagation zone is divided into an internal zone and an external zone, of which the internal zone is configured for ray tracing the eye with the laser beams parallel to the optical axis while the external zone is configured for oblique ray tracing of the eye;
a set of optical elements is placed in the external zone directing the probing laser beams at skew angles;
the structure of each of said first frequency generator and said second frequency generator comprises:
a first oscillator of a first frequency, a second oscillator of a second frequency, a third oscillator of a third frequency, a fourth oscillator of a fourth frequency, and a combination-frequency unit,
said first frequency and said second frequency having a first frequency shift between them that defines a spatial splitting in a first double-beam,
said third frequency and said fourth frequency having a second frequency shift between them that defines a spatial splitting in a second double-beam,
said combination-frequency unit combining said first frequency shift and said second frequency shift as a difference therebetween or as a sum thereof, and
a configuration of the first frequency generator and the second frequency generator enabling a mode of all oscillators functioning simultaneously or a mode of a single functioning oscillator;
a coherent detector is placed on the path of the back scattered laser radiation after the first beam splitter with an aperture in front of said coherent detector;
a low-pass filter is placed at the output of the coherent detector, said low-pass filter being connected to the processing unit; and
a phase discriminator is placed after the low-pass filter with its signal input connected to the output of said low-pass filter, and its reference input having when-enabled connections to the combination-frequency units of said first frequency generator or said second frequency generator, the output of said phase discriminator connected to said processing unit;
said processing unit in its connections with said first frequency generator and said second frequency generator are configured to provide measurement in said first plane and said second plane separately to:
enable the connection of said first frequency, said second frequency, said third frequency and said fourth frequency from said first frequency generator to said first driver, the connection of a single frequency from said second frequency generator to said second driver, the connection of the combination frequency from said first frequency generator to the reference input of said phase discriminator when providing the measurement in the first plane, and
enable the connection of said first frequency, said second frequency, said third frequency and said fourth frequency from said second frequency generator to said second driver, the connection of a single frequency from said first frequency generator to said first driver, the connection of the combination frequency from said second frequency generator to the reference input of said phase discriminator when providing the measurement in the second plane.

7. The device of claim 6, wherein each of said first frequency generator and said second frequency generator are configured as a first four-channel frequency synthesizer and a second four-channel frequency synthesizer with synchronous direct digital synthesis.

8. The device of claim 6, wherein said first frequency generator and said second frequency generator are configured as a single eight-channel frequency synthesizer with synchronous direct digital synthesis, wherein its first four channels function as said first frequency generator, and its second four channels function as said second frequency generator.

9. The device of claim 6, wherein said set of optical elements placed in said external zone comprises a first set of mirrors and a second set of mirrors, each of said set of mirrors consisting of two outward reflecting mirrors, symmetrically placed in reference to the optical axis, and two symmetrically placed inward reflecting mirrors, said first set of mirrors configured to bend the optical paths in said first plane, said second set of mirrors configured to bend the optical paths in said second plane.
